(19) United States
(12) Patent Application Publication
(10) Pub. No.: US 2026/0041386 A1
(43) Pub. Date: Feb. 12, 2026

(54) COMPACT MOVE-AWAY SOLUTION FOR A GANTRY

(71) Applicant: GE Precision Healthcare LLC, Waukesha, WI (US)

(72) Inventors: Romain Boucherie, Paris (FR); Florent Mallet, Paris (FR); Laure Laporte, Morangis (FR); Benjamin Wimille, Versailles (FR); Ali Zakaria, Guyancourt (FR); Benoît Bargain, Villepreux (FR)

(21) Appl. No.: 18/797,998

(22) Filed: Aug. 8, 2024

(51) Int. Cl. A61B 6/00; A61B 6/04; A61B 6/46

(52) U.S. Cl. CPC A61B 6/4441; A61B 6/0407; A61B 6/4405; A61B 6/461; A61B 6/469; A61B 6/547; A61B 6/4464

(57) ABSTRACT

An X-ray imaging system includes an X-ray radiation source and an X-ray detector disposed on a gantry structure. The X-ray imaging system includes a controller configured to perform actions. The actions include defining a virtual wall within a room that the X-ray imaging system is disposed within and receiving a selection of a predefined position to move the gantry structure to from a start position where the gantry structure is located, wherein at the start position the gantry structure is disposed adjacent to or about a patient table for imaging a subject with the X-ray imaging system, and wherein the predefined position is parallel to the virtual wall. The actions include receiving an activation signal to move the gantry structure from the start position to the predefined position in a compact motion and moving the gantry structure from the start position to the predefined position in the compact motion.

### BACKGROUND

The subject matter disclosed herein relates to X-ray imaging systems and, more particularly, to a compact move-away solution for a gantry of an X-ray imaging system.

Medical diagnostic imaging systems generate images of an object, such as a patient, for example, through exposure to an energy source, such as X-rays passing through a patient, for example. The generated images may be used for many purposes. Often, when a practitioner takes X-rays of a patient, it is desirable to take several X-rays of one or more portions of the patient's body from a number of different positions and angles, and preferably without needing to frequently reposition the patient. To meet this need, C-arm X-ray diagnostic equipment has been developed. The term C-arm generally refers to an X-ray imaging device having a rigid and/or articulating structural member having an X-ray source and an image detector assembly that are each located at an opposing end of the structural member so that the X-ray source and the image detector face each other. The structural member is typically “C” shaped and so is referred to as a C-arm. In this manner, X-rays emitted from the X-ray source can impinge on the image detector and provide an X-ray image of the object or objects that are placed between the X-ray source and the image detector.

Sometimes an X-ray imaging system is disposed within an examination room with small dimensions. In certain situation, it might be desirable to move the X-ray imaging system out of the area where the patient is located (e.g., on a patient table) in order to perform a particular task. However, the small dimensions of the examination room may limit the movement of the X-ray imaging system, thus, making it difficult to accomplish the task

### SUMMARY

Certain embodiments commensurate in scope with the originally claimed subject matter are summarized below. These embodiments are not intended to limit the scope of the claimed subject matter, but rather these embodiments are intended only to provide a brief summary of possible forms of the subject matter. Indeed, the subject matter may encompass a variety of forms that may be similar to or different from the embodiments set forth below.

In accordance with an embodiment, an X-ray imaging system is provided. The X-ray imaging system includes an X-ray radiation source. The X-ray imaging system also includes an X-ray detector. The X-ray imaging system further includes a gantry structure. The gantry structure includes a C-arm having the X-ray radiation source disposed on a first end and the X-ray detector disposed on a second end opposite the first end. The C-arm is configured to rotate about multiple different axes. The X-ray imaging system further includes a controller. The controller includes a memory encoding processor executable routines. The controller also includes a processing system including one or more processors and configured to access the memory and to execute the processor-executable routines, wherein the processor-executable routines, when executed by the processing system, cause the processing system to perform actions. The actions include defining a virtual wall within a room that the X-ray imaging system is disposed within. The actions also include receiving, via a user interface, a selection of a predefined position to move the gantry structure to from a start position where the gantry structure is located, wherein at the start position the gantry structure is disposed adjacent to or about a portion of a patient table for imaging a subject with the X-ray imaging system, and wherein the predefined position is parallel to the virtual wall. The actions further include receiving, via the user interface, an activation signal to move the gantry structure from the start position to the predefined position in a compact motion, wherein the compact motion is a motion that is direct as possible in moving the gantry structure without the gantry structure touching or crossing the virtual wall. The actions even further include moving the gantry structure from the start position to the predefined position in the compact motion.

In accordance with another embodiment, a computer-implemented method for moving an X-ray imaging system in a small room is provided. The computer-implemented method includes defining, via a processing system including one or more processors, a virtual wall within a room that the X-ray imaging system is disposed within. The X-ray imaging system includes an X-ray radiation source. The X-ray imaging system also includes an X-ray detector. The X-ray imaging system further includes a gantry structure. The gantry structure includes a C-arm having the X-ray radiation source disposed on a first end and the X-ray detector disposed on a second end opposite the first end. The C-arm is configured to rotate about multiple different axes. The computer-implemented method also includes receiving, at the processing system, from a user interface a selection of a predefined position to move the gantry structure to from a start position where the gantry structure is located, wherein at the start position the gantry structure is disposed adjacent to or about a portion of a patient table for imaging a subject with the X-ray imaging system, and wherein the predefined position is parallel to the virtual wall. The computer-implemented method further includes receiving, at the processing system, from the user interface an activation signal to move the gantry structure from the start position to the predefined position in a compact motion, wherein the compact motion is a motion that is direct as possible in moving the gantry structure without the gantry structure touching or crossing the virtual wall. The computer-implemented method even further includes moving, via the processing system, the gantry structure from the start position to the predefined position in the compact motion.

In accordance with a further embodiment, a non-transitory computer-readable medium is provided. The computer-readable medium includes processor-executable code that when executed by a processing system including one or more processors, causes the processing system to perform actions. The actions include defining a virtual wall within a room that an X-ray imaging system is disposed within. The X-ray imaging system includes an X-ray radiation source. The X-ray imaging system also includes an X-ray detector. The X-ray imaging system further includes a gantry structure. The gantry structure includes a C-arm having the X-ray radiation source disposed on a first end and the X-ray detector disposed on a second end opposite the first end. The C-arm is configured to rotate about multiple different axes. The actions also include receiving, via a user interface, a selection of a predefined position to move the gantry structure to from a start position where the gantry structure is located, wherein at the start position the gantry structure is disposed adjacent to or about a portion of a patient table for imaging a subject with the X-ray imaging system, and wherein the predefined position is parallel to the virtual wall. The actions further include receiving, via the user interface, an activation signal to move the gantry structure from the start position to the predefined position in a compact motion, wherein the compact motion is a motion that is direct as possible in moving the gantry structure without the gantry structure touching or crossing the virtual wall. The actions even further include moving the gantry structure from the start position to the predefined position in the compact motion.

### BRIEF DESCRIPTION OF THE DRAWINGS

These and other features, aspects, and advantages of the present disclosed subject matter will become better understood when the following detailed description is read with reference to the accompanying drawings in which like characters represent like parts throughout the drawings, wherein:

FIG. 1 is a block diagram illustrating components of an example mobile X-ray imaging system, in accordance with aspects of the present disclosure;

FIG. 2 is a block diagram illustrating components of an example an X-ray imaging system (e.g., mounted to a ceiling), in accordance with aspects of the present disclosure;

FIG. 3 is a schematic diagram of a side view of a portion of a mobile X-ray imaging system and its associated movements, in accordance with aspects of the present disclosure;

FIG. 4 is a schematic diagram of a side view of an X-ray imaging system mounted to a ceiling, in accordance with aspects of the present disclosure;

FIG. 5 is a flow chart of a method for moving an X-ray imaging system in a small room, in accordance with aspects of the present disclosure;

FIG. 6 is a schematic diagram of virtual walls defined in a room flanking sides of a patient table (e.g., parallel with the patient table), in accordance with aspects of the present disclosure;

FIG. 7 is a schematic diagram of a virtual wall defined adjacent a longitudinal end of a patient table (e.g., perpendicular with the patient table), in accordance with aspects of the present disclosure;

FIG. 8 is a flow chart of a method for manually moving an X-ray imaging system in a small room, in accordance with aspects of the present disclosure;

FIG. 9 is a schematic diagram of a user interface on a display illustrating multiple predefined positions for selection, in accordance with aspects of the present disclosure;

FIG. 10 is a schematic diagram of a user interface on a display illustrating compact movement of a gantry structure from a start position to a predefined position, in accordance with aspects of the present disclosure;

FIG. 11 is a table of intermediate positions for a move-away to a predefined position, in accordance with aspects of the present disclosure;

FIG. 12 is a schematic diagram of an intermediate position of a gantry structure, in accordance with aspects of the present disclosure; and

FIG. 13 is a schematic diagram of a user interface on a display during manual movement of a gantry structure, in accordance with aspects of the present disclosure.

### DETAILED DESCRIPTION

One or more specific embodiments will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

When introducing elements of various embodiments of the present subject matter, the articles “a,” “an,” “the,” and “said” are intended to mean that there are one or more of the elements. The terms “comprising,” “including,” and “having” are intended to be inclusive and mean that there may be additional elements other than the listed elements. Furthermore, any numerical examples in the following discussion are intended to be non-limiting, and thus additional numerical values, ranges, and percentages are within the scope of the disclosed embodiments.

The present disclosure provides a compact move-away (e.g., drive-away in case of mobile X-ray imaging system) solution for a gantry structure of an X-ray imaging system. In certain embodiments, the X-ray imaging system is a mobile X-ray imaging system including a mobile base (e.g., automated guide vehicle). In certain embodiments, the X-ray imaging system is a X-ray imaging system with the gantry structure mounted on a ceiling within a room or to a floor of the room. The compact move-away solution enables the gantry structure to be moved away from a patient table even in situations where the room is not large enough to move back the gantry structure. For example, a virtual wall is defined along and adjacent one or more walls of the room where that dimension (and the space in the area) is limited. The gantry structure is moved from a start position (e.g., adjacent the patient table) to a predefined position (e.g., parked position) adjacent to the virtual wall. The gantry structure is moved utilizing a compact motion that does not cross or exceed the virtual wall and that does not touch the virtual wall. The gantry structure may be returned to the start position from the predefined position utilizing a motion inverse to the compact motion. The compact-move away solution frees up space adjacent the patient table when needed, even in a small or narrow examination room. The disclosed techniques may also be utilized with an X-ray imaging system that is fixed to floor but the gantry structure has multiple axes of movement.

The disclosed embodiments include an X-ray imaging system that includes an X-ray radiation source and an X-ray detector. The X-ray imaging system also includes a gantry structure. The gantry structure includes a C-arm having the X-ray radiation source disposed on a first end and the X-ray detector disposed on a second end opposite the first end. The C-arm is configured to rotate about multiple different axes. The X-ray imaging system further includes a controller. The controller includes a memory encoding processor executable routines. The controller also includes a processing system including one or more processors and configured to access the memory and to execute the processor-executable routines, wherein the processor-executable routines, when executed by the processing system, cause the processing system to perform actions. The actions include defining a virtual wall within a room that the X-ray imaging system is disposed within. The actions also include receiving, via a user interface, a selection of a predefined position to move the gantry structure to from a start position where the gantry structure is located, wherein at the start position the gantry structure is disposed adjacent to or about a portion of a patient table for imaging a subject with the X-ray imaging system, and wherein the predefined position is parallel to the virtual wall. The actions further include receiving, via the user interface, an activation signal to move the gantry structure from the start position to the predefined position in a compact motion, wherein the compact motion is a motion that is direct as possible in moving the gantry structure without the gantry structure touching or crossing the virtual wall. The actions even further include moving the gantry structure from the start position to the predefined position in the compact motion.

In certain embodiments, the actions include automatically recording the start position. In certain embodiments, the actions include receiving, via the user interface, a user command to move the gantry structure back to the start position (e.g., utilizing the recorded start position) and moving the gantry structure back to the start position from the predefined position in another compact motion that is inverse to the compact motion.

In certain embodiments, the actions include displaying the compact motion on a display for viewing by a user to anticipate the motion. In certain embodiments, the actions include displaying when a user manually moves, via the user interface, the gantry structure a user-perceptible indication that movement of the gantry structure is blocked by the virtual wall and/or the gantry structure is approaching the virtual wall.

In certain embodiments, the gantry structure is mounted to a ceiling of the room. In certain embodiments, both the virtual wall and the predefined position are perpendicular with a longitudinal axis of the patient table.

In certain embodiments, the compact motion includes a motion including translation combined with rotation about a point, wherein the point is defined so that during the compact motion the gantry structure does not cross or touch the virtual wall. In certain embodiments, the X-ray imaging system comprises a mobile base (e.g., automated guided vehicle) coupled to the gantry structure, wherein the mobile base is configured to move the gantry structure. In certain embodiments, wherein the compact motion is performed in multiple steps. In certain embodiments, the compact motion includes first moving the gantry structure to an intermediate position and then performing the motion that includes the translation combined with the rotation about the point to move the gantry structure to the predefined position. In certain embodiments, the actions include selecting the intermediate position from a plurality of predefined intermediate positions based on both a distance between the patient table and the virtual wall and an angle between a first longitudinal axis of the patient table and a second longitudinal axis of the gantry structure at the start position. In certain embodiments, the start position is defined as a projection on a horizontal plane of an isocenter of the gantry structure and the angle. In certain embodiments, both the virtual wall and the predefined position is parallel with the patient table when the room is limited in space adjacent sides of the patient table flanking a longitudinal axis of the patient table. In certain embodiments, both the virtual wall and the predefined position is perpendicular to the patient table when the room is limited in space adjacent the longitudinal ends of the patient table.

The disclosed embodiments include a computer-implemented method for moving an X-ray imaging system in a small room. The method includes defining, via a processing system including one or more processors, a virtual wall within a room that the X-ray imaging system is disposed within. The X-ray imaging system includes an X-ray radiation source. The X-ray imaging system also includes an X-ray detector. The X-ray imaging system further includes a gantry structure. The gantry structure includes a C-arm having the X-ray radiation source disposed on a first end and the X-ray detector disposed on a second end opposite the first end. The C-arm is configured to rotate about multiple different axes. The method also includes receiving, at the processing system, from a user interface a selection of a predefined position to move the gantry structure to from a start position where the gantry structure is located, wherein at the start position the gantry structure is disposed adjacent to or about a portion of a patient table for imaging a subject with the X-ray imaging system, and wherein the predefined position is parallel to the virtual wall. The method further includes receiving, at the processing system, from the user interface an activation signal to move the gantry structure from the start position to the predefined position in a compact motion, wherein the compact motion is a motion that is direct as possible in moving the gantry structure without the gantry structure touching or crossing the virtual wall. The method even further includes moving, via the processing system, the gantry structure from the start position to the predefined position in the compact motion.

In certain embodiments, the method includes automatically recording, via the processing system, the start position. In certain embodiments, the method includes receiving, at the processing system from the user interface, a user command to move the gantry structure back to the start position. In certain embodiments, the method includes moving, via the processing system, the gantry structure back to the start position from the predefined position in another compact motion that is inverse to the compact motion.

The disclosed embodiments include a non-transitory computer-readable medium. The computer-readable medium includes processor-executable code that when executed by a processing system including one or more processors, causes the processing system to perform actions. The actions include defining a virtual wall within a room that an X-ray imaging system is disposed within. The X-ray imaging system includes an X-ray radiation source. The X-ray imaging system also includes an X-ray detector. The X-ray imaging system further includes a gantry structure. The gantry structure includes a C-arm having the X-ray radiation source disposed on a first end and the X-ray detector disposed on a second end opposite the first end. The C-arm is configured to rotate about multiple different axes. The actions also include receiving, via a user interface, a selection of a predefined position to move the gantry structure to from a start position where the gantry structure is located, wherein at the start position the gantry structure is disposed adjacent to or about a portion of a patient table for imaging a subject with the X-ray imaging system, and wherein the predefined position is parallel to the virtual wall. The actions further include receiving, via the user interface, an activation signal to move the gantry structure from the start position to the predefined position in a compact motion, wherein the compact motion is a motion that is direct as possible in moving the gantry structure without the gantry structure touching or crossing the virtual wall. The actions even further include moving the gantry structure from the start position to the predefined position in the compact motion.

In certain embodiments, the actions include automatically recording, via the processing system, the start position. In certain embodiments, the actions include receiving, at the processing system from the user interface, a user command to move the gantry structure back to the start position. In certain embodiments, the actions include moving, via the processing system, the gantry structure back to the start position from the predefined position in another compact motion that is inverse to the compact motion.

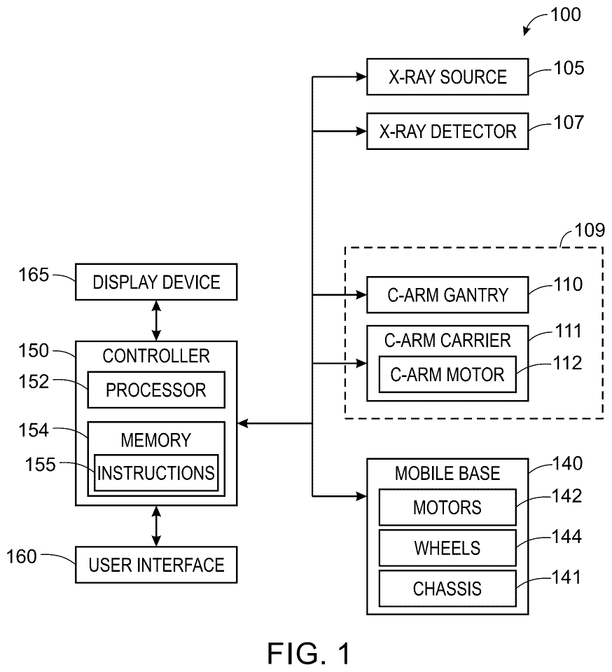

FIG. 1 is a block diagram illustrating components of an example mobile X-ray imaging system 100. In certain embodiments, the mobile X-ray imaging system 100 is configured to perform interventional imaging (e.g., vascular imaging). The mobile X-ray imaging system 100 includes an X-ray radiation source 105 and an X-ray detector 107 mounted on a gantry structure 109, in particular, a C-arm gantry 110 (e.g., C-arm).

The gantry structure 109 includes a C-arm motor 112 for adjusting the position of the C-arm gantry 110. More specifically, the C-arm gantry 110 is mechanically coupled to a C-arm carrier 111 (e.g., C-arm rotation device) which includes the C-arm motor 112, and the C-arm motor 112 may be driven to adjust the position of the C-arm gantry 110 with respect to the C-arm carrier 111. For example, the C-arm carrier 111 in conjunction with the C-arm motor 112 is configure to rotate the C-arm gantry 110 in an orbital direction relative to the C-arm carrier 111. In certain embodiments, the C-arm carrier 111 (via a motorized system) is configured to rotate a pivot (e.g., pivot point) where the C-arm carrier 111 is coupled to a mobile base 140 (e.g. automated guided vehicle) or an end of an L-arm coupled to the mobile base 140. The C-arm carrier 111 rotates about a rotational axis (e.g., horizontal axis) of the pivot. In certain embodiments having an L-arm, L-arm may rotate about a location where the other of the L-arm (i.e., the end of the L-arm not connected to the pivot) is coupled to the mobile base 140.

The mobile X-ray imaging system 100 also includes the mobile base 140. The C-arm carrier 111 is coupled to the mobile base 140. The mobile base 140 is configured to move (e.g., translocate) the mobile X-ray imaging system 100 from one location to another location on a floor. The mobile base 140 includes a chassis 141. The mobile base 140 includes one or more motors 142 for driving one or more wheels 144 (e.g. drive wheels) to adjust a position of the mobile base 140. In addition, one or more of the wheels 144 may be free or un-motorized.

The mobile X-ray imaging system 100 further includes a controller 150 including a processor 152 and a non-transitory memory 154. A method for controlling the mobile X-ray imaging system 100 may be stored as executable instructions 155 in the non-transitory memory 154 and executed by the processor 152.

The mobile X-ray imaging system 100 further include a user interface 160 for receiving input from a user or operator of the mobile X-ray imaging system 100. The user interface 160 may be communicatively coupled to the controller 150 for providing commands input by a user via the user interface 160 to the controller 150. The user interface 160 may include one or more of a keyboard, a mouse, a trackball, one or more knobs, one or more joysticks, a touchpad, a touchscreen, one or more hard and/or soft buttons, a smartphone, a microphone, a virtual reality apparatus, and so on. The user interface 160 may thus enable voice control, and display of information such as an interactive display device (e.g., touchscreen). In certain embodiments, the user may manually move the mobile X-ray imaging system 100 via the user interface 160. In some examples the user interface 160 may be remotely located relative to the mobile X-ray imaging system 100. For example, the user interface 160 may be communicatively coupled to the controller 150 and/or the mobile X-ray imaging system 100 via a wired or wireless connection, and may be positioned away from the mobile base 140.

As an example, the memory 154 may store processor-executable software code or instructions (e.g., firmware or software), which are tangibly stored on a non-transitory computer readable medium. Additionally or alternatively, the memory 154 may store data. As an example, the memory 154 may include a volatile memory, such as random access memory (RAM), and/or a nonvolatile memory, such as read-only memory (ROM), flash memory, a hard drive, or any other suitable optical, magnetic, or solid-state storage medium, or a combination thereof. Furthermore, the processor 152 may include multiple microprocessors, one or more “general-purpose” microprocessors, one or more special-purpose microprocessors, and/or one or more application specific integrated circuits (ASICS), or some combination thereof. For example, the processor 152 may include one or more reduced instruction set (RISC) or complex instruction set (CISC) processors. The processor 152 may include multiple processors, and/or the memory 154 may include multiple memory devices.

A user of the mobile X-ray imaging system 100 may input a desired isocenter position via the user interface 160, for example. The controller 150 may then determine position adjustments to one or more of the C-arm gantry 110 and the mobile base 140 to align an isocenter of the mobile X-ray imaging system 100 with the desired isocenter position. As another example, a user of the mobile X-ray imaging system 100 may directly control the position of one or more components of the mobile X-ray imaging system 100 relative to other components of the mobile X-ray imaging system 100 via the user interface 160. For example, the user may directly input, via a joystick or knob, for example, position adjustments to one or more components of the mobile X-ray imaging system 100. As another example, the motion of the components of the mobile X-ray imaging system 100 may be pre-programmed such that the user does not directly control any movement, but instead initiates the start of the pre-programmed motion. The motion may include complex motions, with continuous motion of the isocenter.

As described in greater detail below, the controller 150 may be configured to utilize a compact move-away solution for the gantry structure 109 of the mobile X-ray imaging system 100. The compact move-away solution enables the gantry structure 109 to be moved away from a patient table even in situations where the room is not large enough to move back the gantry structure 109. For example, a virtual wall is defined along and adjacent one or more walls of the room where that dimension (and the space in the area) is limited. The gantry structure 109 is moved from a start position (e.g., adjacent the patient table) to a predefined position (e.g., parked position) adjacent to the virtual wall. The gantry structure 109 is moved utilizing a compact motion that does not cross or exceed the virtual wall and that does not touch the virtual wall. The gantry structure 109 may be returned to the start position from the predefined position utilizing a motion inverse to the compact motion. The compact-move away solution frees up space adjacent the patient table when needed, even in a small or narrow examination room.

The controller 150 is further communicatively coupled to a display device 165 for displaying one or more X-ray images acquired via the X-ray detector 107. Further, in some examples, one or more of the controller 150, the user interface 160, and the display device 165 may be positioned away from (e.g., remotely from) the remaining components of the mobile X-ray imaging system 100.

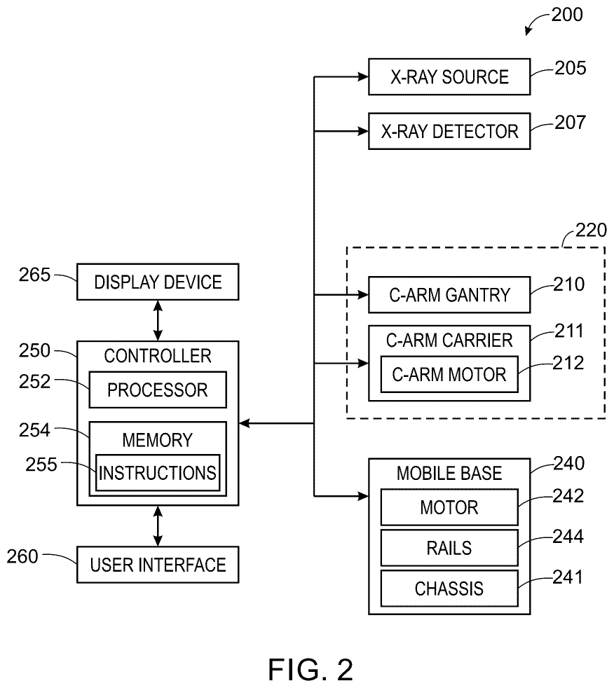

FIG. 2 is a block diagram illustrating components of an example an X-ray imaging system 200 (e.g., mounted to a ceiling). In certain embodiments, the X-ray imaging system 200 is configured to perform interventional imaging (e.g., vascular imaging). The X-ray imaging system 200 is configured to be mounted to a ceiling. In certain embodiments, the X-ray imaging system 200 is mounted to the ceiling In certain embodiments, the X-ray imaging system 200 is mounted to the ceiling but may move along the ceiling (e.g., via chassis that moves along a rail system or other type of system for movement). The X-ray imaging system 200 includes an X-ray radiation source 205 and an X-ray detector 207 mounted on a C-arm gantry 210 (e.g., C-arm).

The C-arm gantry 210 is part of a gantry structure 220. The gantry structure 220 includes a C-arm motor 212 for adjusting the position of the C-arm gantry 210. More specifically, the C-arm gantry 210 is mechanically coupled to a C-arm carrier 211 (e.g., C-arm rotation device) which includes the C-arm motor 212, and the C-arm motor 212 may be driven to adjust the position of the C-arm gantry 210 with respect to the C-arm carrier 211. For example, the C-arm carrier 211 in conjunction with the C-arm motor 212 is configured to rotate the C-arm gantry 210 in an orbital direction relative to the C-arm carrier 211. In certain embodiments, the C-arm carrier 211 (via a motorized system) is configured to rotate a pivot (e.g., pivot point) where the C-arm carrier 211 is coupled to an end of an arm (e.g., L-arm) coupled to the ceiling. The C-arm carrier 211 rotates about a rotational axis (e.g., horizontal axis) of the pivot. In certain embodiments, the C-arm carrier 211 is coupled to or mounted to the ceiling.

In certain embodiments, the X-ray imaging system 200 also includes a mobile base 240. The mobile base 240 is coupled to a ceiling. In certain embodiments, the C-arm carrier 211 is coupled to the mobile base 240 via an L-arm (e.g., via the end of the L-arm not connected to the pivot). In certain embodiments, the C-arm carrier 211 is coupled to the mobile base 240. The mobile base 240 is configured to move (e.g., translocate) the X-ray imaging system 200 from one location to another location (e.g., in a linear direction) on the ceiling. The mobile base 240 includes a chassis 241. The mobile base 240 also includes a motor 242 and a rail system 244 (e.g., having rails). The rail system 244 is directly coupled to the ceiling. The motor 242 is configured to drive movement of the chassis 241 and, thus, the X-ray imaging system 200 along the rail system 244 (e.g., to adjust a position of the X-ray imaging system 200). In certain embodiments, the X-ray imaging system 200 may include a different system from a rail and chassis to move the X-ray imaging system 200.

The X-ray imaging system 200 further includes a controller 250 including a processor 252 and a non-transitory memory 254. A method for controlling the X-ray imaging system 200 may be stored as executable instructions 155 in the non-transitory memory 254 and executed by the processor 252.

The X-ray imaging system 200 further include a user interface 260 for receiving input from a user or operator of the X-ray imaging system 200. The user interface 260 may be communicatively coupled to the controller 250 for providing commands input by a user via the user interface 260 to the controller 250. The user interface 260 may include one or more of a keyboard, a mouse, a trackball, one or more knobs, one or more joysticks, a touchpad, a touchscreen, one or more hard and/or soft buttons, a smartphone, a microphone, a virtual reality apparatus, and so on. The user interface 260 may thus enable voice control, and display of information such as an interactive display device (e.g., touchscreen). In some examples the user interface 260 may be remotely located relative to the X-ray imaging system 200. For example, the user interface 260 may be communicatively coupled to the controller 250 and/or the X-ray imaging system 200 via a wired or wireless connection, and may be positioned away from the mobile base 240.

As an example, the memory 254 may store processor-executable software code or instructions (e.g., firmware or software), which are tangibly stored on a non-transitory computer readable medium. Additionally or alternatively, the memory 254 may store data. As an example, the memory 254 may include a volatile memory, such as random access memory (RAM), and/or a nonvolatile memory, such as read-only memory (ROM), flash memory, a hard drive, or any other suitable optical, magnetic, or solid-state storage medium, or a combination thereof. Furthermore, the processor 252 may include multiple microprocessors, one or more “general-purpose” microprocessors, one or more special-purpose microprocessors, and/or one or more application specific integrated circuits (ASICS), or some combination thereof. For example, the processor 252 may include one or more reduced instruction set (RISC) or complex instruction set (CISC) processors. The processor 252 may include multiple processors, and/or the memory 254 may include multiple memory devices.

A user of the X-ray imaging system 200 may input a desired isocenter position via the user interface 260, for example. The controller 250 may then determine position adjustments to one or more of the C-arm gantry 210 and/or the mobile base 240 to align an isocenter of the X-ray imaging system 200 with the desired isocenter position. As another example, a user of the X-ray imaging system 200 may directly control the position of one or more components of the X-ray imaging system 200 relative to other components of the X-ray imaging system 200 via the user interface 260. For example, the user may directly input, via a joystick or knob, for example, position adjustments to one or more components of the X-ray imaging system 200. As another example, the motion of the components of the X-ray imaging system 200 may be pre-programmed such that the user does not directly control any movement, but instead initiates the start of the pre-programmed motion. The motion may include complex motions, with continuous motion of the isocenter.

As described in greater detail below, the controller 250 may configured to utilize a compact move-away solution for the gantry structure 220 of the X-ray imaging system 200 (e.g., mounted to the ceiling). The compact move-away solution enables the gantry structure 220 to be moved away from a patient table even in situations where the room is not large enough to move back the gantry structure 220. For example, a virtual wall is defined along and adjacent one or more walls of the room where that dimension (and the space in the area) is limited. The gantry structure 220 is moved from a start position (e.g., adjacent the patient table) to a predefined position (e.g., parked position) adjacent to the virtual wall. The gantry structure 220 is moved utilizing a compact motion that does not cross or exceed the virtual wall and that does not touch the virtual wall. The gantry structure 220 may be returned to the start position from the predefined position utilizing a motion inverse to the compact motion. The compact-move away solution frees up space adjacent the patient table when needed, even in a small or narrow examination room.

The controller 250 is further communicatively coupled to a display device 265 for displaying one or more X-ray images acquired via the X-ray detector 207. Further, in some examples, one or more of the controller 250, the user interface 260, and the display device 265 may be positioned away from (e.g., remotely from) the remaining components of the X-ray imaging system 200.

The X-ray imaging system 200 may further include a cooling system 268 for cooling the X-ray radiation source 205 and/or the X-ray detector 207. The cooling system 268 may include one or more flexible tubes and a pump, as an illustrative and non-limiting example, for providing cooling fluid to the X-ray radiation source 205 to transfer thermal energy away from the X-ray radiation source 205. The cooling system 268 may actively cool the X-ray radiation source 205 and the X-ray detector 207 independently, or in some examples may cool the X-ray detector 207 by any suitable type of derivation of the cooling circuit for the X-ray radiation source 205.

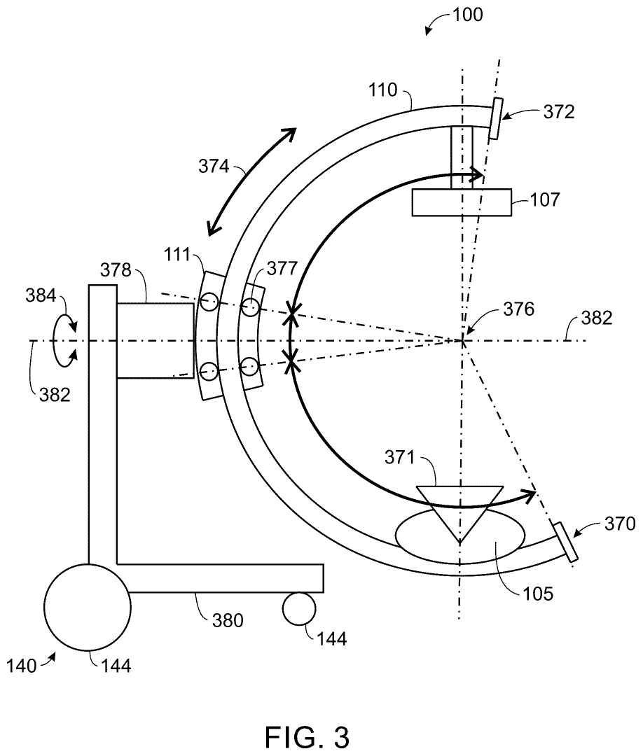

FIG. 3 is a schematic diagram of a side view of a portion of the mobile X-ray imaging system 100 and its associated movements. The mobile X-ray imaging system 100 includes the C-arm gantry 110. The mobile X-ray imaging system 100 also includes the X-ray radiation source 105 coupled to a first end 370 of the C-arm gantry 110 and the X-ray detector 107 coupled to a second end 372 of the C-arm gantry 110 opposite the first end 370. A collimator 371 is coupled to the X-ray radiation source 105 and is configured to collimate the X-ray beam.

The C-arm gantry 110 is coupled to the C-arm carrier 111 (e.g., C-arm rotation device) is configure to rotate the C-arm gantry 110 in an orbital direction 374 relative to the C-arm carrier 111 about an isocenter 376 (of the X-ray radiation source 105 and the X-ray detector 107). The C-arm carrier 111 includes rollers 377 (e.g., guiding rollers) to guide movement of the C-arm gantry 110 relative to the C-arm carrier 111.

The C-arm carrier 111 is coupled to a pivot 378 (e.g., pivot point or shaft). The pivot 378 is coupled to a structure 380. The pivot 378 (in conjunction with a motorized system) is configured to rotate both the C-arm carrier 111 and the C-arm gantry 110 about a rotational axis 382 (e.g., horizontal axis) of the pivot 378 as indicated by arrow 384. As depicted, the structure 380 is the mobile base 140 as depicted in FIG. 3. The mobile base 140 includes wheels 144 to move the mobile X-ray imaging system 100 along a floor. In certain embodiments, the structure 380 is an L-arm coupled to the mobile base 140. In certain embodiments, the mobile X-ray imaging system 100 lacks the mobile base 140 and is fixed to the floor.

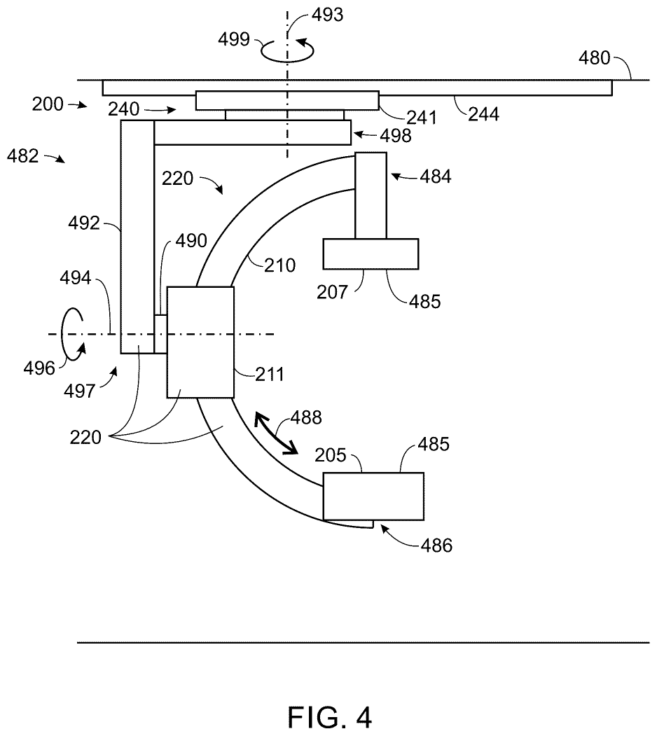

FIG. 4 is a schematic diagram of a side view of the X-ray imaging system 200 (e.g., having an L-arm) mounted to a ceiling 480 (e.g., in a room 482). The X-ray imaging system 200 includes the C-arm gantry 210. The X-ray imaging system 200 also includes the X-ray detector 207 coupled to a first end 484 of the C-arm gantry 210 and the X-ray radiation source 205 coupled to a second end 486 of the C-arm gantry 210 opposite the first end 484 (e.g. forming the image chain 485).

The C-arm gantry 210 is coupled to the C-arm carrier 211 (e.g., C-arm rotation device) is configured to rotate the C-arm gantry 210 in an orbital direction 488 relative to the C-arm carrier 211 about an isocenter (of the X-ray radiation source 205 and the X-ray detector 207). The C-arm carrier 211 includes rollers (e.g., guiding rollers) to guide movement of the C-arm gantry 210 relative to the C-arm carrier 211.

The C-arm carrier 211 is coupled to a pivot 490 (e.g., pivot point or shaft). The pivot 490 is coupled to a structure 492. The pivot 490 (in conjunction with a motorized system) that is configured to rotate both the C-arm carrier 211 and the C-arm gantry 210 about a rotational axis 494 (e.g., horizontal axis) of the pivot 490 as indicated by arrow 496. In certain embodiments, the structure 492 is an L-arm coupled to the mobile base 240. The pivot 490 is coupled to a first end 497 of the L-arm and mobile base 240 is coupled to a second end 498 of the L-arm. As depicted, the gantry structure 220 is coupled to the mobile base 240 (thus, mounting the X-ray imaging system 200 to the ceiling 480). In certain embodiments, the L-arm may rotate about an end of the L-arm coupled to the mobile base 240. In particular, the L-arm (and the gantry structure 220) rotate in direction 499 about a rotational axis 493. The chassis 241 (driven by a motor) is configured to move the X-ray imaging system 200 along the rail system 244.

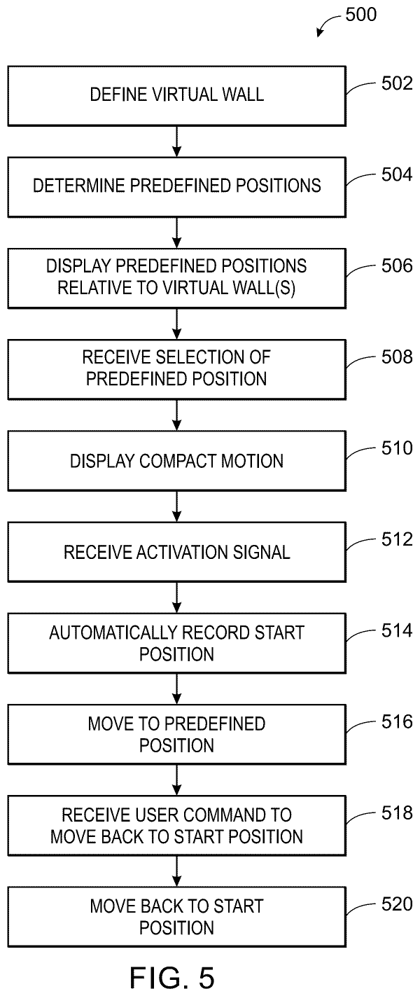

FIG. 5 is a flow chart of a method 500 for moving an X-ray imaging system (e.g., mobile X-ray imaging system 100 in FIG. 1 or X-ray imaging system 200 in FIG. 2) in a small room. One or more steps of the method 500 may be performed by one or more components of the medical imaging system (e.g., controller 150 in FIG. 1 or controller 250 in FIG. 2). One or more steps of the method 500 may be performed simultaneously and/or in a different order from that depicted in FIG. 5.

The method 500 includes defining a virtual wall within a room that the X-ray imaging system is disposed within (block 502). In certain embodiments, two virtual wall may be defined. The virtual wall is defined based on the dimensions in the room. In particular, the virtual wall is disposed within the room adjacent to and running parallel with a wall (or walls) defining the smallest dimension (e.g., width or length) out of the dimensions in the room.

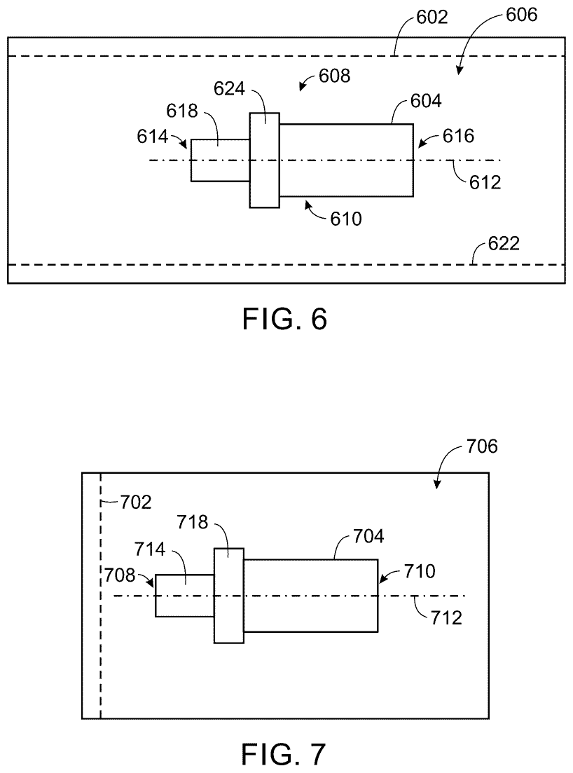

In certain embodiments, the virtual wall (e.g., virtual wall 602) is parallel with a patient table 604 in a room 606 when the room is limited in space adjacent sides 608, 610 of the patient table 604 flanking a longitudinal axis 612 of the patient table 604 as depicted in FIG. 6. The patient table 604 includes longitudinal ends 614, 616. The patient table 604 includes a table top 618 (e.g., cradle) configured to support a subject (e.g., patient to be imaged). As depicted in FIG. 6, two virtual wall 602, 622 are defined (e.g., to the left and right of the patient table 604 relative to the longitudinal axis 612). The imaging system (in particular, the gantry structure) is represented by rectangle 624 in FIG. 6.

In certain embodiments, the virtual wall (e.g., virtual wall 702) is perpendicular with a patient table 704 in a room 706 when the room 706 is limited in space adjacent the longitudinal ends 708, 710 of the patient table 704 (e.g., relative to a longitudinal axis 712 of the patient table 704) as depicted in FIG. 7. The patient table 704 includes a table top 714 (e.g., cradle) configured to support a subject (e.g., patient to be imaged). The imaging system (in particular, the gantry structure) is represented by rectangle 718 in FIG. 7. In certain embodiments, when the X-ray imaging system is mounted to the ceiling, the virtual wall 702 (e.g., depicted adjacent longitudinal end 708 of the patient table 704) may only be perpendicular relative the longitudinal axis 712 of the patient table 704. In certain embodiments, a virtual wall may also be established adjacent longitudinal end 710 of the patient table 704. In certain embodiments, a virtual wall may parallel to the patient table 704. In certain embodiments, more than two virtual walls may be utilized.

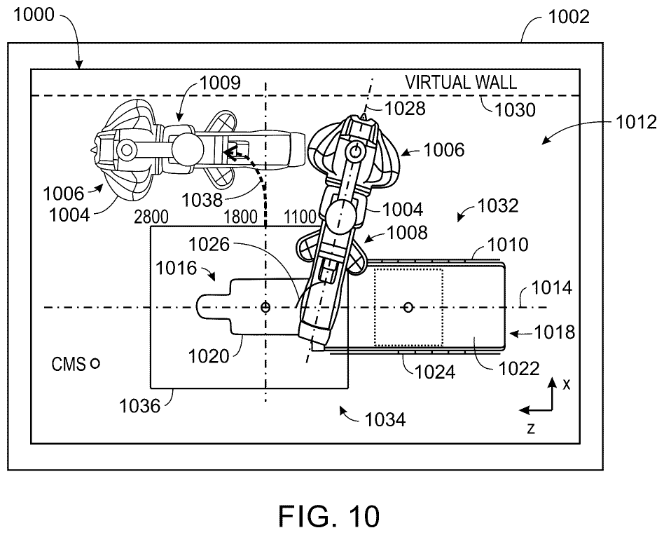

Returning to FIG. 5, the method 500 includes determining one or more predefined positions (e.g., parked positions) for moving a gantry structure to from a start position where the gantry structure is located (block 504). Each predefined position is parallel with and adjacent to a virtual wall. At the start position, the gantry structure is disposed adjacent to or about a portion of a patient table for imaging a subject with the X-ray imaging system. The start position is defined as a projection on a horizontal of plane of an isocenter (e.g., isocenter 376 in FIG. 3) of the gantry structure (i.e., center of rotations of its axes) and an angle (e.g., swivel angle) between a longitudinal axis of the patient table and a longitudinal axis of the gantry structure (see FIG. 10) at the start position.

The method 500 also includes displaying the one or more predefined positions relative to one or more virtual walls (block 506). The predefined positions and virtual walls may be displayed on a user interface (e.g., user interface 160 in FIG. 1 or user interface 260 in FIG. 2) on a display (e.g., display device 165 in FIG. 1 or display device 265 in FIG. 2).

The method 500 further includes receiving, from a user interface, a selection of a predefined position (e.g., from among the one or more predefined positions) to move the gantry structure to from the start position where the gantry structure is located (block 508). The method 500 even further includes displaying a compact motion of the gantry structure from the start position to the selected predefined position on a display (e.g., user interface) for viewing by a user to anticipate the motion (block 510).

The method 500 still further includes receiving, from the user interface, an activation signal to move the gantry structure from the start position to the selected predefined position in a compact motion (block 512). The compact motion is a motion that is direct as possible in moving the gantry structure without the gantry structure touching the virtual wall and without the gantry structure crossing the virtual wall. In certain embodiments, the compact motion includes a motion including a translation combined with rotation about the point, where the point is defined so that during the compact motion, the gantry structure does not cross or touch the virtual wall. In certain embodiments, the compact motion is performed in multiple steps (e.g., for a mobile X-ray imaging system). In this scenario, the compact motion first includes moving the gantry structure to an intermediate position and then performing the motion that includes the translation combined with the rotation about the point to move the gantry structure to the selected predefined position. In certain embodiments, the controller selects an intermediate position from a plurality of predefined intermediate positions based on both a distance between the patient table and the virtual wall and the angle (e.g., the swivel angle) between the longitudinal axis of the patient table and the longitudinal axis of the gantry structure at the start position. In certain embodiments (e.g., with the X-ray imaging system mounted to the ceiling), one predefined move-away position is proposed at the head (e.g. longitudinal end) of the patient table.

The method 500 includes automatically recording the start position (block 514). The method 500 also includes moving the gantry structure from the start position to the predefined position in the compact motion (block 516). The method 500 further includes receiving, via the user interface, a user command to move the gantry structure back to the start position (e.g. selection of start position from among other positions) (block 518). The method 500 even further includes moving the gantry structure back to the start position from the predefined position in another compact motion that is inverse to the original compact motion (to move the gantry structure form the start position to the selected predefined position) (block 520).

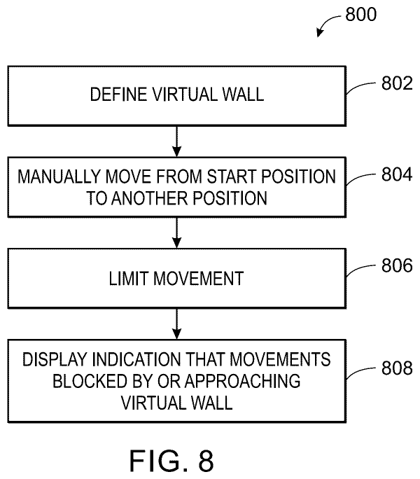

FIG. 8 is a flow chart of a method 800 for moving an X-ray imaging system (e.g., mobile X-ray imaging system 100 in FIG. 1 or X-ray imaging system 200 in FIG. 2) manually in a small room. One or more steps of the method 800 may be performed by one or more components of the medical imaging system (e.g., controller 150 in FIG. 1 or controller 250 in FIG. 2). One or more steps of the method 800 may be performed simultaneously and/or in a different order from that depicted in FIG. 8. The method 800 may occur at any point subsequent to block 502 in the method 500 in FIG. 5.

The method 800 includes defining a virtual wall within a room that the X-ray imaging system is disposed within (block 802). In certain embodiments, two virtual wall may be defined. The virtual wall is defined based on the dimensions in the room. In particular, the virtual wall is disposed within the room adjacent to and running parallel with a wall (or walls) defining the smallest dimension (e.g., width or length) out of the dimensions in the room. The one or more virtual walls may be as described in FIGS. 6 and 7.

The method 800 also includes a user manually moving the gantry structure, utilizing the user interface, from the start position to another position (block 804). The method 800 further includes utilizing the virtual wall to limit motions (and/or block movement) of the gantry structure when the gantry is translated or rotated directly (i.e., manually) by the user via the user interface (block 806). The method 800 even further includes displaying a user-perceptible indication on a display (e.g., user interface) that movement of the gantry structure is limited and/or blocked by the virtual wall and/or that the gantry structure is approaching the virtual wall (block 808).

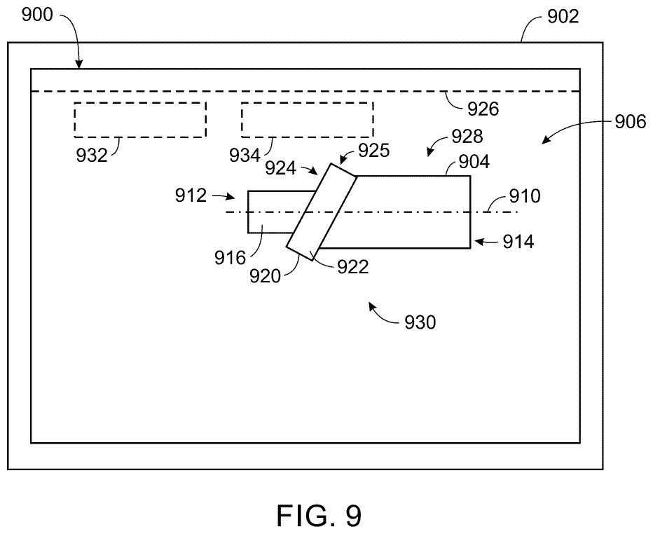

FIG. 9 is a schematic diagram of a user interface 900 (e.g., for viewing by the user) on a display 902 illustrating multiple predefined positions for selection. The user interface 900 for presenting the possible predefined positions for selection may vary from that shown in FIG. 9.

As depicted, a patient table 904 and its position within a room 906 is shown in the user interface 900. The patient table 904 includes a longitudinal axis 910 and longitudinal ends 912, 914. The patient table 904 includes a table top 916 (e.g., cradle) configured to support a subject (e.g., patient to be imaged). A rectangle 920 represents a gantry structure 922 of an X-ray imaging system 924 (e.g., mobile X-ray imaging system) in a start position 925 (the gantry structure's current position).

A virtual wall 926 is also shown that extends parallel to the longitudinal axis 910 on a side 928 of the patient table 904. In certain embodiments, the virtual wall 926 may be shown on the opposite side 930 of the patient table 904 or an additional virtual wall may be shown on the opposite side of the patient table 904. In certain embodiments, the virtual wall 926 may be shown perpendicular to the longitudinal axis 910 of the patient table 904 adjacent one of the longitudinal ends 912, 914.

Predefined positions for the moving the gantry structure 922 are shown adjacent to and parallel with the virtual wall 926 by dashed rectangles 932, 934. The user may select from the plurality of predefined positions 932, 934. For examples, the user may select one of the predefined positions 932, 934 via a user input device (e.g., via touching the user interface 900 (e.g., display 902) or using another input device). In certain embodiments, the predefined positions 932, 934 may each be labeled and a list may be presented to the user on the user interface 900 with the labels for the user to select a particular predefined position 932, 934.

FIG. 10 is a schematic diagram of a user interface 1000 on a display 1002 illustrating compact movement of a gantry structure 1004 (e.g., of a mobile X-ray imaging system 1006) from a start position 1008 to a predefined position 1009 (e.g., upon selection of the predefined position 1009). The user interface 1000 for displaying the compact motion may vary from that shown in FIG. 10.

As depicted, a patient table 1010 and its position within a room 1012 is shown in the user interface 1000. The patient table 1010 includes a longitudinal axis 1014 and longitudinal ends 1016, 1018. The patient table 1010 includes a table top 1020 (e.g., cradle) configured to support a subject (e.g., patient to be imaged) that can be extended to and from a base 1022 of the patient table 1010 via a rail system 1024.

As depicted, in the start position 1008, the gantry structure 1004 is disposed at an angle 1026 (e.g., swivel angle) about a portion of the patient table 1010. The angle 1026 is defined between a longitudinal axis 1028 of the gantry structure 1004 and the longitudinal axis 1014 of the patient table 1010.

A virtual wall 1030 is also shown that extends parallel to the longitudinal axis 1014 on a side 1032 of the patient table 1010. In certain embodiments, the virtual wall 1030 may be shown on the opposite side 1034 of the patient table 1010 or an additional virtual wall may be shown on the opposite side of the patient table 1010 (see FIG. 6). In certain embodiments, the virtual wall 1030 may be shown perpendicular to the longitudinal axis 1014 of the patient table 1010 adjacent one of the longitudinal ends 1016, 1018.

In certain embodiments (e.g., with the mobile X-ray imaging system 1006), movement of the gantry structure 1004 from the start position 1008 to the predefined position 1009 occurs in multiple steps (e.g., two steps). In the first step, the gantry structure 1004 is moved to an intermediate position within an area 1036 (e.g., adjacent the patient table 1010 and the start position 1008). A user can control or modify movement of the gantry structure 1004 within this area 1036 (e.g., by releasing the activation signal). Outside of the area 1036, the user cannot manually control or modify movement of the gantry structure 1004. Outside of the area 1036, it is only possible to move to the predefined position or move back to the intermediate position. In certain embodiments, instead of returning the gantry structure 1004 back to the start position 1008 from the predefined position a user may select a different return position but the gantry structure will still first move to the intermediate position.

Upon moving from the start position 1008 to the intermediate position (i.e., the first step), a compact motion combining rotation and translation (represented by arrow 1038) to move the gantry structure 1004 from the intermediate position to the predefined position 1009 is performed (i.e., step 2). The two steps are linked and performed without additional action from the user. The gantry structure 1004 may be returned from the predefined position 1009 to the start position 1008 by performing the steps in reverse (e.g., with an inverse motion).

In certain embodiments (e.g., with the mobile X-ray imaging system 1006 having an automated guided vehicle), two symmetrical predefined move-away positions may be proposed with one being to the left of the patient table 1010 (i.e., side 1032) when facing the longitudinal end 1016 and one to right of the patient table 1010 (i.e., side 1034). The system adapts the motion trajectory to the start position 1008 of the gantry structure 1004 and to the size of the room 1012. The start position 1008 is defined as the projection on a horizontal plane (e.g., parallel with the floor of the room) of the isocenter (e.g., isocenter 376 in FIG. 3) of the gantry structure 1004 (i.e., center of rotations of its axes) and the angle 1026. The intermediate position is selected (e.g., by the controller) among a list of predefined intermediate positions based on the value (e.g., swivel value) of the angle 1026 at the start position 1008 and a distance in x-direction between the virtual wall 1030 and the patient table 1010 (e.g., the longitudinal axis 1014 of the patient table 1010).

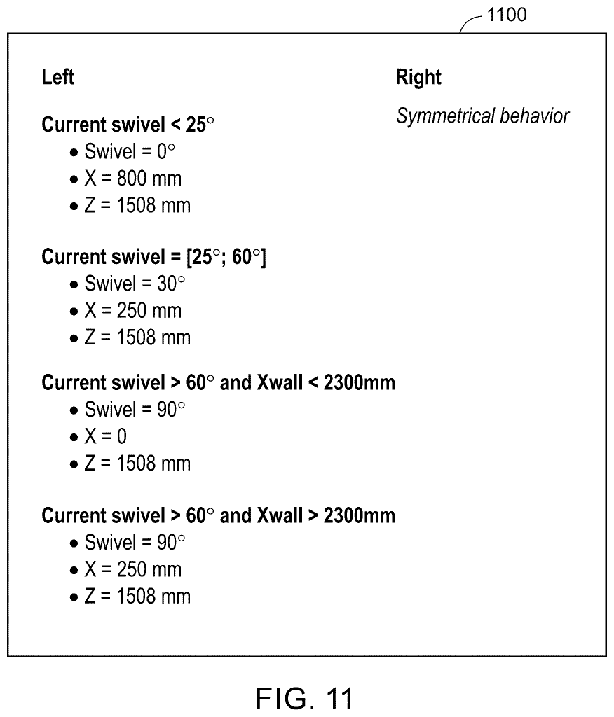

FIG. 11 is a table 1100 of intermediate positions for a move-away to a predefined position (e.g., predefine position 1009 in FIG. 10) to a left (e.g., side 1032 in FIG. 10) of the patient table (e.g., patient table 1010 in FIG. 10). Multiple predefined intermediate positions are listed in the table for movement to the left based on the angle (e.g., angle 1026 in FIG. 10) and the distance (e.g., referred to as Xwall in the table 1100) in the x-direction (see FIG. 10) between the virtual (e.g., virtual wall 1030) and longitudinal axis of the patient table in FIG. 10. The listed intermediate positions are examples and many more intermediate positions may be possible. Intermediate position for move-away to a predefined position to the right of the patient table may occur in a symmetrical manner to those for the left. As depicted in the table 1100, intermediate positions are shown for swivel angles of 0, 30 and 90 degrees for the move-away to a predefined position to the left. As noted above, a motion combining gantry rotation and translation is executed when moving between the intermediate position and the final position (i.e., predefined position). The combined motion is based on the translation of a point with respect to gantry structure while the gantry structure is rotating around this point. In certain embodiments, other types of combined motion that utilize translation with a rotation may be utilized. The point is defined in such a way that during the motion, the gantry does not exceed the virtual wall and does not touch the virtual wall. For an intermediate position at a swivel angle of 0 degrees, only a translation is performed in the step since the gantry structure is well oriented. In certain embodiments, the movement from a start position to a predefined position (and vice versa) may occur directly without utilizing an intermediate position.

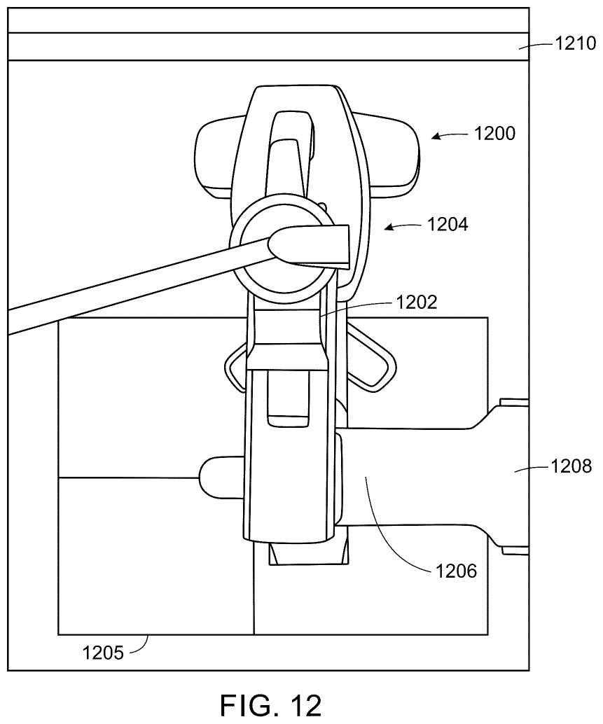

FIG. 12 is a schematic diagram of an intermediate position 1200 of a gantry structure 1202 (e.g., of a mobile X-ray imaging system 1204) with respect to area 1205. As depicted in FIG. 12, the gantry structure 1202 is disposed about a portion of a table top 1206 (e.g., patient cradle) of a patient table 1208. The intermediate position 1200 of the gantry structure 1202 has a current swivel angle of greater than 60 degrees and a virtual wall 1210 is located less than 2300 millimeters (mm) from the patient table 1208 in the x-direction. In particular, the intermediate position 1200 of the gantry structure 1202 has a swivel angle of 90 degrees and the isocenter is located 0 mm from the longitudinal axis of the table 1208 in the x-direction and at 1508 mm from a table reference center (e.g., center point of the table 1208) in the z-direction.

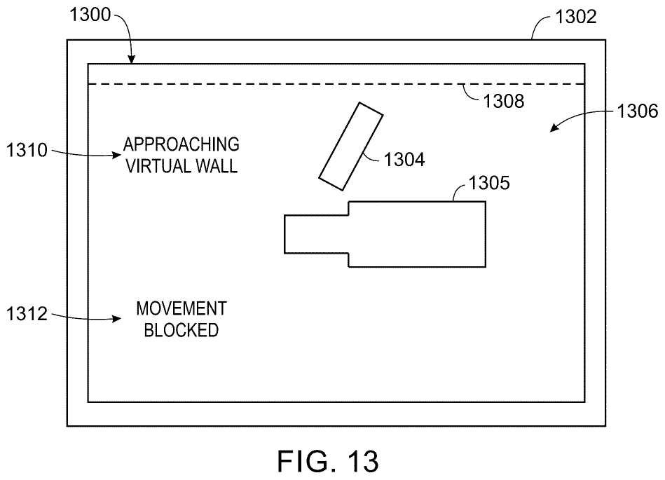

FIG. 13 is a schematic diagram of a user interface 1300 on a display 1302 during manual movement of a gantry structure 1304. The presentation of the user interface 1300 may vary from that depicted in FIG. 13. The user interface 1300 depicts the position of the patient table 1305 within the room 1306. The user interface 1300 also depicts the current position of the gantry structure 1304 relative to a virtual wall 1308. User-perceptible indications 1310, 1312 are depicted on the user interface 1300 indicating the gantry structure 1304 is approaching the virtual wall 1308 and that movement of the gantry structure 1304 is blocked. As depicted, the indications 1310, 1312 are textual indications. In certain embodiments, the representation of the gantry structure 1304 and/or the virtual wall 1308 may flash or change color (e.g., from a first color (e.g., yellow) to a second color (e.g., red) different from the first color to provide a warning of possibly touching or crossing the virtual wall 1308. In certain embodiments, a sound (e.g., beep) or alarm may be provided via a speaker on the imaging system as the indication or warning.

Technical effects of the disclosed embodiments include providing a compact move-away (e.g., drive-away in case of mobile X-ray imaging system) solution for a gantry structure of an X-ray imaging system. Technical effects of the disclosed embodiments also include enabling the gantry structure to be moved away from a patient table even in situations where the room is not large enough to move back the gantry structure. For example, a virtual wall is defined along and adjacent one or more walls of the room where that dimension (and the space in the area) is limited. The gantry structure is moved from a start position (e.g., adjacent the patient table) to a predefined position (e.g., parked position) adjacent to the virtual wall. The gantry structure is moved utilizing a compact motion that does not cross or exceed the virtual wall and that does not touch the virtual wall. The gantry structure may be returned to the start position from the predefined position utilizing a motion inverse to the compact motion. The compact-move away solution frees up space adjacent the patient table when needed, even in a small or narrow examination room.

The techniques presented and claimed herein are referenced and applied to material objects and concrete examples of a practical nature that demonstrably improve the present technical field and, as such, are not abstract, intangible or purely theoretical. Further, if any claims appended to the end of this specification contain one or more elements designated as “means for [perform]ing [a function] . . . ” or “step for [perform]ing [a function] . . . ”, it is intended that such elements are to be interpreted under 35 U.S.C. 112(f). However, for any claims containing elements designated in any other manner, it is intended that such elements are not to be interpreted under 35 U.S.C. 112(f).

This written description uses examples to disclose the present subject matter, including the best mode, and also to enable any person skilled in the art to practice the subject matter, including making and using any devices or systems and performing any incorporated methods. The patentable scope of the subject matter is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

1. An X-ray imaging system, comprising:
an X-ray radiation source;
an X-ray detector;
a gantry structure comprising:
a C-arm having the X-ray radiation source disposed on a first end and the X-ray detector disposed on a second end opposite the first end, wherein the C-arm is configured to rotate about multiple different axes; and
a controller comprising:
a memory encoding processor-executable routines; and
a processing system comprising one or more processors and configured to access the memory and to execute the processor-executable routines, wherein the processor-executable routines, when executed by the processing system, cause the processing system to:
define a virtual wall within a room that the X-ray imaging system is disposed within;
receive, via a user interface, a selection of a predefined position to move the gantry structure to from a start position where the gantry structure is located, wherein at the start position the gantry structure is disposed adjacent to or about a portion of a patient table for imaging a subject with the X-ray imaging system, and wherein the predefined position is parallel to the virtual wall;
receive, via the user interface, an activation signal to move the gantry structure from the start position to the predefined position in a compact motion, wherein the compact motion is a motion that is direct as possible in moving the gantry structure without the gantry structure touching or crossing the virtual wall; and
move the gantry structure from the start position to the predefined position in the compact motion.

2. The X-ray imaging system of claim 1, wherein the processor-executable routines, when executed by the processing system, cause the processing system to automatically record the start position.

3. The X-ray imaging system of claim 2, wherein the processor-executable routines, when executed by the processing system, cause the processing system to:
receive, via the user interface, a user command to move the gantry structure back to the start position; and
move the gantry structure back to the start position from the predefined position in another compact motion that is inverse to the compact motion.

4. The X-ray imaging system of claim 1, wherein the processor-executable routines, when executed by the processing system, cause the processing system to display the compact motion on a display for viewing by a user.

5. The X-ray imaging system of claim 1, wherein the processor-executable routines, when executed by the processing system, cause the processing system, when a user manually moves, via the user interface, the gantry structure, to utilize the virtual wall to limit or block movement of the gantry structure and to display a user-perceptible indication that movement of the gantry structure is limited or blocked by the virtual wall and/or the gantry structure is approaching the virtual wall.

6. The X-ray imaging system of claim 1, wherein the gantry structure is mounted to a ceiling of the room or to a floor of the room.

7. The X-ray imaging system of claim 6, wherein both the virtual wall and the predefined position are perpendicular with a longitudinal axis of the patient table.

8. The X-ray imaging system of claim 6, wherein the both the virtual wall and the predefined position are perpendicular with a longitudinal axis of the patient table.

9. The X-ray imaging system of claim 1, wherein the X-ray imaging system comprises a mobile base coupled to the gantry structure, wherein the mobile base is configured to move the gantry structure.

10. The X-ray imaging system of claim 1, wherein the compact motion comprises a motion including translation combined with rotation about a point, wherein the point is defined so that during the compact motion the gantry structure does not cross or touch the virtual wall.

11. The X-ray imaging system of claim 10, wherein the compact motion is performed in multiple steps.

12. The X-ray imaging system of claim 11, wherein the compact motion comprises first moving the gantry structure to an intermediate position and then performing the motion that includes the translation combined with the rotation about the point to move the gantry structure to the predefined position.

13. The X-ray imaging system of claim 12, wherein the processor-executable routines, when executed by the processing system, cause the processing system to select the intermediate position from a plurality of predefined intermediate positions based on both a distance between the patient table and the virtual wall and an angle between a first longitudinal axis of the patient table and a second longitudinal axis of the gantry structure at the start position.

14. The X-ray imaging system of claim 13, wherein the start position is defined as a projection on a horizontal plane of an isocenter of the gantry structure and the angle.

15. The X-ray imaging system of claim 10, wherein both the virtual wall and the predefined position is parallel with the patient table when the room is limited in space adjacent sides of the patient table flanking a longitudinal axis of the patient table.

16. The X-ray imaging system of claim 10, wherein both the virtual wall and the predefined position is perpendicular to the patient table when the room is limited in space adjacent longitudinal ends of the patient table.

17. A computer-implemented method for moving an X-ray imaging system in a small room, comprising:
defining, via a processing system comprising one or more processors, a virtual wall within a room that the X-ray imaging system is disposed within, wherein the X-ray imaging system comprises:
an X-ray radiation source;
an X-ray detector;
a gantry structure comprising:
a C-arm having the X-ray radiation source disposed on a first end and the X-ray detector disposed on a second end opposite the first end, wherein the C-arm is configured to rotate about multiple different axes;
receiving, at the processing system, from a user interface a selection of a predefined position to move the gantry structure to from a start position where the gantry structure is located, wherein at the start position the gantry structure is disposed adjacent to or about a portion of a patient table for imaging a subject with the X-ray imaging system, and wherein the predefined position is parallel to the virtual wall;
receiving, at the processing system, from the user interface an activation signal to move the gantry structure from the start position to the predefined position in a compact motion, wherein the compact motion is a motion that is direct as possible in moving the gantry structure without the gantry structure touching or crossing the virtual wall; and
moving, via the processing system, the gantry structure from the start position to the predefined position in the compact motion.

18. The computer-implemented method of claim 17, further comprising: automatically
recording, via the processing system, the start position;
receiving, at the processing system from the user interface, a user command to move the gantry structure back to the start position; and
moving, via the processing system, the gantry structure back to the start position from the predefined position in another compact motion that is inverse to the compact motion.

19. A non-transitory computer-readable medium, the non-transitory computer-readable medium comprising processor-executable code that when executed by a processing system comprising one or more processors, causes the processing system to:
define a virtual wall within a room that an X-ray imaging system is disposed within, wherein the X-ray imaging system comprises:
an X-ray radiation source;
an X-ray detector;
a gantry structure comprising:
a C-arm having the X-ray radiation source disposed on a first end and the X-ray detector disposed on a second end opposite the first end, wherein the C-arm is configured to rotate about multiple different axes;
receive, via a user interface, a selection of a predefined position to move the gantry structure to from a start position where the gantry structure is located, wherein at the start position the gantry structure is disposed adjacent to or about a portion of a patient table for imaging a subject with the X-ray imaging system, and wherein the predefined position is parallel to the virtual wall;
receive, via the user interface, an activation signal to move the gantry structure from the start position to the predefined position in a compact motion, wherein the compact motion is a motion that is direct as possible in moving the gantry structure without the gantry structure touching or crossing the virtual wall; and
move the gantry structure from the start position to the predefined position in the compact motion.

20. The non-transitory computer-readable medium of claim 19, wherein the processor-executable code, when executed by the processing system, causes the processing system to:
automatically record the start position;
receive, via the user interface, a user command to move the gantry structure back to the start position; and
move the gantry structure back to the start position from the predefined position in another compact motion that is inverse to the compact motion.
